(19) United States
(12) Patent Application Publication
(10) Pub. No.: US 2008/0208479 A1
(43) Pub. Date: Aug. 28, 2008

(54) Devices and Methods for Calculating and Communicating Differences in Measured Data

(75) Inventor: Michael A. Roes, San Diego, CA (US)

Correspondence Address: CATALYST LAW GROUP, APC, 9710 SCRANTON ROAD, SUITE S-170, SAN DIEGO, CA, 92121, US

(73) Assignee: INCLINE TECHNOLOGIES, INC., San Diego, CA (US)

(21) Appl. No.: 11/722,991

(22) PCT Filed: Feb. 7, 2006
(86) PCT No.: PCT/US06/04370

§ 371 Date: Jun. 27, 2007

Related U.S. Application Data
(60) Provisional application No. 60/651,354, filed on Feb. 8, 2005.

(51) Int. Cl. G01G 9/00; G01N 33/00

(52) U.S. Cl. 702/19; 702/173

(57) ABSTRACT

The current invention provides a measurement device having a data processing means that collects measurement data points, stores the collected measured data points and calculates differences and changes in said measurement data points. The device communicates these changes in measured data points to the user. The device does not communicate the measured value to a user. Also provided is a method for monitoring changes in measured data.

### FIELD OF THE INVENTION

This invention relates generally to the field of devices and methods for measuring various aspects of the human body, such as weight, body mass, fat percentage, bioimpedience, calorie count and other similar information. These devices and methods collect and analyze measured data to communicate reports of relative differences between measurements within said collection.

### BACKGROUND

There are currently many types of bathroom scales which display trend information. However, these scales actually provide too much information for many users, and in all devices of the prior art, actual weight is displayed. This is despite the fact that many people experience undue psychological stress when informed of their actual weight. In fact, in the pursuit of weight loss, the actual weight is largely immaterial. More important is the trend information and the knowledge that a certain diet strategy is or isn't working.

In U.S. Pat. No. 3,967,690, issued to Northcutt, there is described a scale for use in a personal dieting program, comprising: weight measuring means; means for storing a previous measurement of weight for comparison with a subsequent measurement; and a digital display capable of displaying both said subsequent weight measured by said scale and the difference between said subsequent weight and said previous weight measured. U.S. Pat. No. 4,318,447, also issued to Northcutt, describes a digital scale that is capable of displaying a variety of measurements, including the amount of weight change necessary to reach an inputted weight goal. The device displays the number of pounds above or below a user is from a desired weight. In U.S. Pat. No. 4,301,879 issued to Dubrow, there is described a weight measuring device which shows historical data by presenting absolute weight points as a function of time. The data points shown herein include the absolute weight on any given date.

In U.S. Pat. No. 4,423,792, issued to Cowan, there is described a weight measuring device that shows a variety of data points including current weight and calculated weight change from an original input baseline weight.

U.S. Pat. No. 4,629,015, issued to Freid et al., describes a device that monitors actual weight loss during dialysis, and will adjust the dialysis process rate if the total weight loss at a given time is too great. The device displays the start weight (actual weight) and the subsequent weight measurements over time (actual weight loss). The device also displays a preferred weight loss over time and the difference between actual and preferred is compared. If the actual weight loss deviates greatly from the preferred an alarm will sound and the dialysis treatment is then adjusted.

In U.S. Pat. No. 5,839,901, issued to Karkanen, there is described a weight loss management device and method wherein a variety of data are entered into the device for the creation of a weight loss database. The device, when used, displays the actual weight of the user.

U.S. Pat. No. 6,354,996, issued to Drinan et al., describes a body composition analysis device that measures and displays a variety of data types, including weight. In addition to displaying the actual measurements, including weight, the device will also display an historical trend.

U.S. Pat. No. 6,538,215, issued to Montagnino et al., describes a device that receives and displays a variety of data type, including absolute weight, for multiple users. The device separates data between multiple users using a keypad. The device displays current as well as trend data for this variety of users.

The devices in the prior art display the actual weight or other measurement of a user. For many users, however, these values have a negative effect. For example, many overweight individuals do not want to know their absolute weight, but would rather see only their progress in a weight loss program. Progress can have more of a positive effect on a user than can absolute weight, even if less than a prior measurement. For these users, the actual values are not needed. What is important to these users is the progress that is being made.

### SUMMARY OF THE INVENTION

One embodiment of the present invention relates to a device for monitoring the change in weight over a period of time. In this embodiment, the actual weight of the user is never displayed. Rather, what is displayed is a change in weight from earlier time points. Thus, a user is never confronted with their actual weight and the negative impact this value may have and instead will only encounter the progress made.

In one particular aspect of the present invention, a microprocessor receives and stores measurements for a user. These measurements are stored in the microprocessor's memory as a function of time and can further be compared and the difference communicated to the user. In addition, the microprocessor can create a user profile for one or many users, thereby keeping each user's information compartmentalized and inaccessible to use by other persons. Profiles can be password protected or otherwise similarly protected. The user profile contains, for example, information about each user's absolute weight and changes in weight over a period of time.

The microprocessor will compare data points (measurements at a given time) for a user and will communicate the differences between data points to that user. Accumulated data points form a data set and various data points in the data set are compared and communicated to the user as differences between data point A and data point N. Data points A and N are data points obtained from the device during different measurings. The compared data points can be communicated visually as a graph or an absolute difference value, or audibly as a percent change or an absolute number value for the change.

In one embodiment of the method, a user's change in weight over a period of time is displayed. In this method, a user will set up a user profile, which compartmentalizes each user's accumulated data preventing viewing and contamination by other users. In this embodiment, the user will input an initial measurement (reference measurement) into the device. This reference measurement is considered the baseline for generating comparisons with subsequently accumulated data. The display will not display the reference measurement, thus the user has no indication of the absolute value of the measurement (e.g., for a body weight scale—the user will not know his or her absolute weight). During subsequent uses the device will obtain a new measurement data points, will compare the newly obtained data points to the existing data points and will communicate only the comparative results.

### BRIEF DESCRIPTION OF THE DRAWINGS

FIG. 1a shows the device having an external housing and having an electronics component.

FIG. 1b illustrates the electronics component of the current device.

FIG. 2a illustrates the communication of comparative data in absolute value of difference over time in graph format.

FIG. 2b illustrates the communication of comparative data as percentage difference over time in a graph format using a best fit line.

FIG. 2c illustrates the communication comparative data in absolute value of difference over time in bar-graph format.

FIG. 3a illustrates a method of use for the current invention for a single user.

FIG. 3b illustrates a method of use for the current invention for multiple users.

### DETAILED DESCRIPTION OF THE INVENTION

### Abbreviations and Terms

In accordance with the present invention and as used herein, the following terms and abbreviations are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following abbreviations are used herein:

The term “difference data” refers to the difference between currently obtained measurements and previously obtained measurements. The previously obtained measurements can be single data points, collections of data points, or the entire set of data points.

The term “measurement” refers to data that is received from the device relating to a feature of the user. Measured data includes, but is not limited to, body weight, body fat, fat percentage, bioimpedience, calorie count and body mass. Measured data is stored in the device memory as a data point.

The term “reference measurement” refers to the first data point collected for a data set. The reference measurement may be included in the set of data points that are used for comparison with a currently received measured data point when generating the difference data; however, it is not necessarily included. Thus, the reference data is only called reference data herein because it is the first data point for a user's data set.

The term “data point” refers to the data obtained during a single measurement.

The term “data set” refers to all of the data points obtained for a user.

The device of the current invention can be any weighing device, such as weight scales, body mass indicators, fat scales, fat analyzers and combinations thereof; and, for this description of the current invention, the term “device” will be used to refer to these and other similar devices. The preferred embodiment of the device, herein, is that of a bathroom scale for obtaining the weight of a user. Those of ordinary skill in the art will readily apply the current invention to the variety of similar devices, and such applications are well within the spirit of the current invention.

In a preferred embodiment, the device of the current invention is a weight scale useful for obtaining weight measurements in humans. In a further preferred embodiment, the device of the current invention provides a recognition means, such as a password and a keyboard, a key, a biometric indicator, or other similar means. The recognition means is useful for preventing persons other than the user from being measured by the device and having that measurement added to a user's data set. Such an event can be detrimental to the user's data set and in turn will cause the device to display inaccurate difference data to the user. Furthermore, for devices wherein multiple users are measured by a single device, the recognition means allows the device to obtain and compartmentalize measured data for each user. Again, compartmentalization will prevent the mixing of users' data points and in turn the communication of inaccurate difference data.

For embodiments using biometric identification systems, a user's unique biometric identifier, (e.g., fingerprint, iris scan, toe print, etc), is required to set-up, access, review and alter a user profile. Biometric identification means are well known in the art and can be employed with the current invention device by those of ordinary skill in the art. In the preferred embodiment herein, the biometric identifier recognizes the toe-print of a user. This embodiment is preferred because the biometric identifier can be placed on the measuring (weighing) platform of the device such that when the user steps on the device to be measured, the user's toe is in position for biometric scanning and identification. Thus the user is minimally burdened by the recognition means. Nonetheless, other biometric recognition means can be employed with the current invention.

For embodiments using passwords and keypads, it is preferable that the keypad is conveniently placed such that the user can enter the password with minimal burden. Thus, in a preferred embodiment, the keypad is separated from the device, thereby allowing the keypad to be stored on a shelf while the device resides on the floor. The keypad can communicate with the device either wirelessly or via a hard wire connection. With the hard wire connection, it is preferable that the wire is long and flexible thereby allowing for freedom of movement. The keypad can be simple, e.g., a numeric keypad, or can be more complex, e.g., a qwerty keypad.

Other recognition means can be used herein, including, but not limited to, a key.

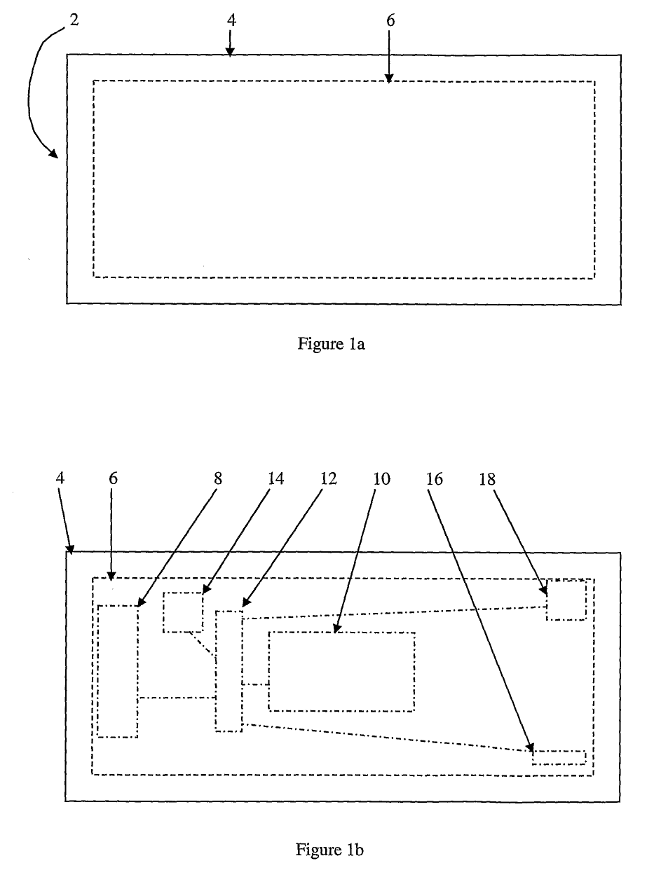

Referring now to FIG. 1a, the device 2 of the current invention is described. In a preferred embodiment, the device 2 of the current invention is a digital bathroom weight scale having a housing 4, and an electronics component 6. The electronics component 6, as shown in FIG. 1b, further comprises, a communication means 8, a measuring means 10 and a data processing means 12. In addition, the electronics component can further comprise a recognition means 14, a programming means 16 and a reset means 18.

In the preferred embodiment, the device 2 is positioned on the floor, and housing 4 is substantially flat such that housing 4 further comprises a platform, or top surface, allowing a user to comfortably stand on said device in the measurement position and be measured by the device. The device 2 will then measure the user using the measuring means 10, which for a weight measuring device can be a strain gauge, load cell or other well know means for obtaining weight. Means for obtaining measurements, including but not limited to, body weight, body fat, fat percentage, bioimpedience, calorie count and body mass, are well known in the art. The device 2 will transfer the measured data to the data processing means 12 where the measured data is stored as a data point. In this regard, the data processing means 12 is preferably a microprocessor with memory that stores and compares data points comprising measurement and date. When the device 2 receives the initial measurement, that measurement is stored as a data point termed the reference measurement. Subsequent measurements are stored as additional data points in the data processing means 12, thereby forming a data set. Individual data points comprising a data set can be compared and any differences in these compared data points can be determined and communicated to the user. Changes in measurement over time between different specific combinations of data points within the data set, or between all data points within the data set can be determined, and these determined differences are referred to collectively as “compared data points.”

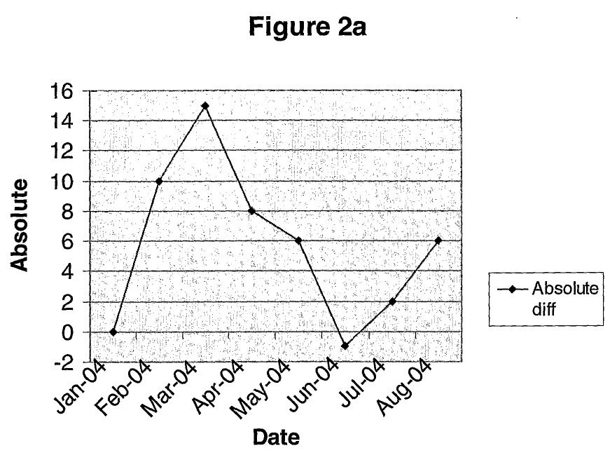

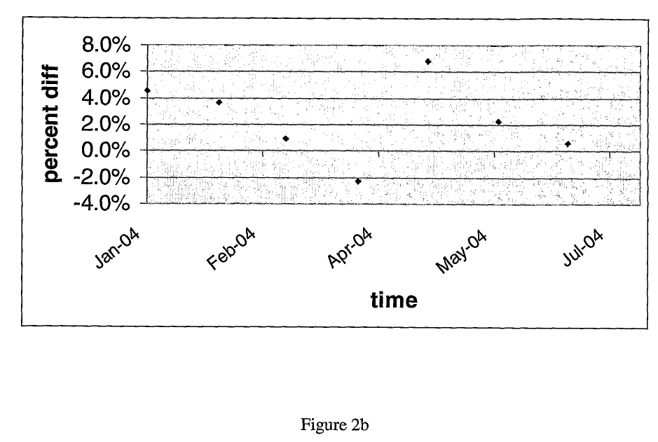

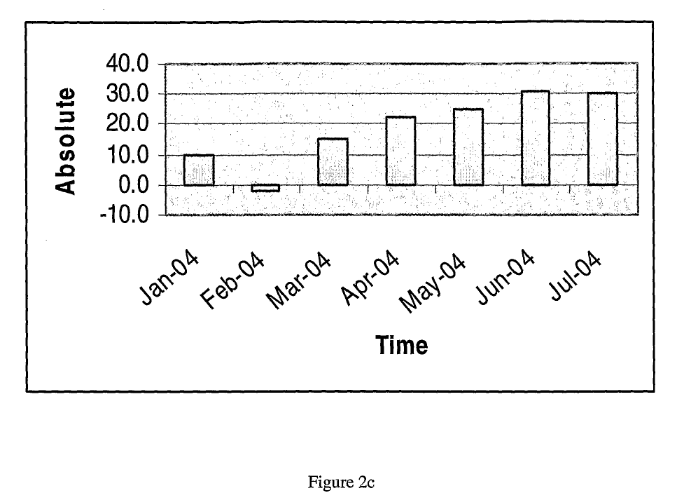

The compared data points are then arranged into a format for communication to the user via the communication means 8. In the preferred embodiment, the communication means 8 is an LCD, and the compared data is presented as a graph having difference between data points on the y-axis and time on the x-axis. FIG. 2a. The difference herein is between the data point derived from the reference measurement and each subsequent measurement beginning at the first subsequent measurement. The line shown is point-to-point; however, a variety of other graph formats can be used including, best fit lines and bar graphs. FIGS. 2b and 2c, respectively. Also, in the example in FIG. 2a, the difference in data points is absolute value; however, other comparison values can be used (e.g., percent difference, etc.).

A variety of different compared data can be communicated to the user in a variety of different forms. By way of example only, the compared data can be the current measurement compared to the just previous measurement and communicated as a single absolute difference value in bar graph format. Alternatively, the compared data can be the most recently obtained data point compared to an earlier data point in the data set representing a milestone and the difference can be verbally communicated to the user. In this particular example, communication means 8 is a speaker. Those of ordinary skill in the art will recognize that measured data can be compared and the differences communicated to the user using a variety of communication means and in a variety of formats.

It is anticipated that a user of device 2 will from time to time want to erase the data set stored in the data processing means 12. To accomplish the erasing of a data set stored in the data processing means 12, the device 2 can further comprise a reset means 18. The reset means 18 will clear all data points stored in data processing means 12, thereby allowing the user to create a new data set. This is desirable for a variety of reasons, including but not limited to, completion of a measurement goal and the later desire to start a new measurement goal for a same user, and completion of use of the device 2 by one user and the start of use of the device 2 by another user.

Preferably the reset means 18 is such that the device is not accidentally reset. This goal can be achieved by a number of means, some of which are presented herein. In one example, the reset means 18 is based on the time that a user is in the measurement position on the device 2. In this example the device 2 allows sufficient time for measuring the user, for storing the data point, for comparing data points within a data set, and for communicating any information to the user. After this sufficient time, the device 2 will indicate to the user that the reset means 18 will be activated if the user does not remove himself or herself from the measurement position. The user will be given an additional time period to comply with this notice. Should the user comply, the reset means 18 will not be activated; however, if the user remains in the measurement position, the reset means 18 will activate.

In another example, the reset means 18 can be a recessed button or a switch. In the preferred embodiment, this recessed button or switch is placed on the housing 4 of device 2 is a position where said button or switch will not be activated accidentally when the user is in the measurement position, or during any other normal course of use (e.g., storing the device and etc.). Using the recessed button to further describe this example, the recessed button can be located on the edge or bottom of the device 2 housing 4 (this assumes that the measuring position takes place on a surface called the top). Thus, when the user is in the measuring position, the user is not on the same housing 4 surface as is the reset means, and thus will not accidentally reset the device 2. If the user decides to reset the device 2, the user will have to actively access the recessed button and depress it. As an added measure of caution, the button can also have a warning indicator that a reset will take place if the button is not released. The warning can be time based as is described above.

The device 2 can be designed as a single user device or as a multiple user device. With a multiple user device there is the added complexity that a particular user's data points have to be obtained, stored, compared and communicated separately from the other users' data points. To be clear, if multiple users are using the device 2, the device must separate each user's data points in order to deliver a user specific communication to any of the users. Mixing of user data will result in the comparisons of data points in a data set to me an inaccurate and useless communication. In this respect, one alternative embodiment for the current invention provides a recognition means 14 for the current invention.

Different types of recognition means 14 include, but are not limited to the following: password and a keyboard; a key; a biometric indicator, or other similar means. The recognition means 14 is useful for preventing persons other than the user from being measured by the device 2 and having that measurement added to a user's data set. Such an event can be detrimental to the user's data set and in turn will cause the device 2 to display inaccurate differences in compared data for the user. Furthermore, for devices 2 wherein multiple users are measured by a single device, the recognition means 14 allows the device 2 to obtain and compartmentalize measured data for each user. Again, compartmentalization will prevent the mixing of user's data points and in turn the communication of inaccurate difference data.

For embodiments using biometric identification system as a recognition means 14 a user's unique biometric identifier, e.g., fingerprint, iris scan, toe print, etc, is required to set-up, access, review and alter a user profile. Biometric identifications means are well known in the art and can be employed with the current invention device by those of ordinary skill in the art. In the preferred embodiment herein, the biometric identifier recognized is the toe-print of a user. This embodiment is preferred because the biometric identifier can be placed on the measuring (weighing) platform of the device 2 such that when the user steps on the device 2 to be measured, the user's toe is in position for biometric scanning and identification. Thus the user is minimally burdened by the recognition means 14. Nonetheless, other biometric recognition means can be employed with the current invention.

For embodiments using passwords and keypads as the recognition means 14, it is preferable that the keypad is conveniently placed such that the user can enter the password with minimal burden. Thus, in a preferred embodiment, the keypad is separated from the device 2, thereby allowing the keypad to be stored on a shelf while the device 2 resides on the floor. The keypad can communicate with the device either wirelessly, via a hard wire connection or by some other means known in the art. With the hard wire connection, it is preferable that the wire is long and flexible thereby allowing freedom of movement. The keypad can be simple, e.g., a numeric keypad, or can be more complex, e.g., a qwerty keypad.

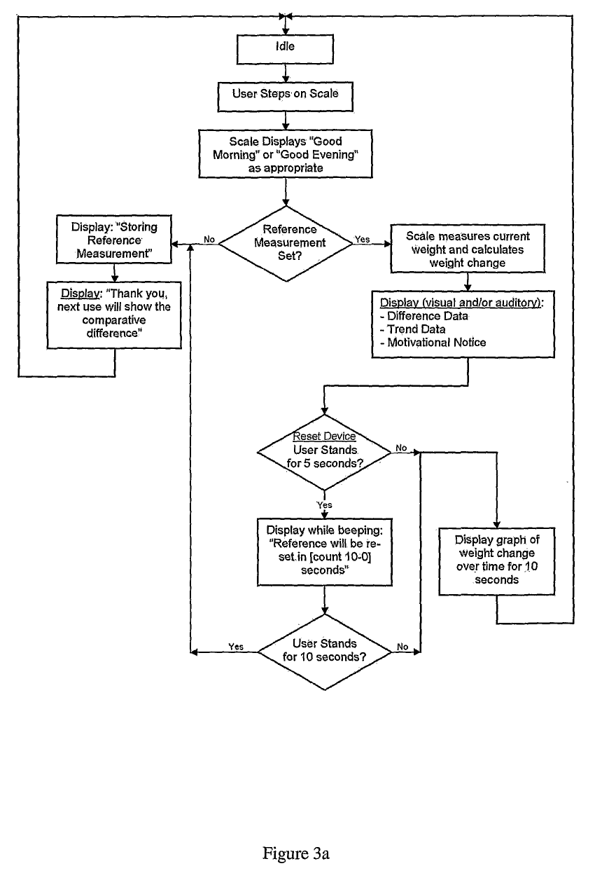

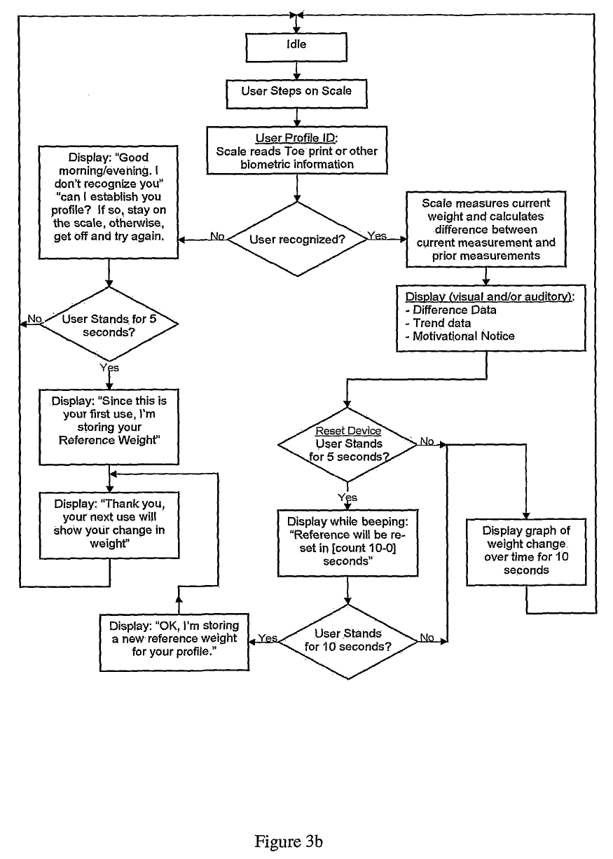

In an alternative embodiment, the device 2 includes a programming means 16. The programming means is useful in many aspects. For example, a weight loss plan can be programmed into device 2 using the program means 16. This weight loss plan can be prescribed by a doctor or can be designed by the user. The plan can include target weight goals and rates of weight loss. The goals can be communicated to the user in a variety of ways including an acknowledgement (e.g., verbal or written message communicated to the user over the communication means 8) or a comparative graph that is displayed at each use. FIG. 3 shows this comparative graph.

Additionally, the programming means can be used to transfer data sets to and from a variety of machines. This is desirable, for example, if a user's weight loss progress is monitored and/or evaluated by a dietician or physician. The history can be presented to the dietician or physician for analysis upon visitation. Changes to the weight loss program can be made by the dietician or physician and saved on a media that is transferred to the user's device 2.

A variety of other data can be transferred to and from the invention device using the programming means 16. The programming means 16 can include, but is not limited to: a port to accept floppy disk, CD media, DVD media, or jump drives; a keyboard and printer, allowing the user to print information on paper and to add information using the keyboard; and broadband, fiber optic, telephone line or wireless communication connection allowing the user to transfer and receive information. In these regards, the device 2 is properly equipped to have such programming means 16, and those of ordinary skill in the art are capable of equipping the device 2 with these and other programming means 16.

### EXAMPLES

Various modifications and alterations of the invention will become apparent to those skilled in the art without departing from the spirit and scope of the invention, which is defined by the accompanying claims. For example, it should be noted that steps recited in any method claims below do not necessarily need to be performed in the order that they are recited. Those of ordinary skill in the art will recognize variations in performing the steps from the order in which they are recited. For example, in certain embodiments, steps may be performed simultaneously. The accompanying claims should be constructed with these principles in mind.

### Example 1

### Single-User Device

In this example, which is illustrated in FIG. 3a, the device 2 of the current invention is a single user weight scale. At the initial stage, meaning that the device has been powered on but the user has not stepped on the scale, the device is considered to be in idle mode. The device can either be powered on by way of being plugged into the wall or the device can operate off of an internal power source. Further, the power on step could have been at the initial measuring, and is left on between that measuring and subsequent measurings, thereby falling into idle mode after a period of non-use. Alternatively, the device can be powered on before each use and powered off following each use.

The user step on the scale to bring the device out of idle mode. Alternatively, the user may flip a switch or otherwise deliver a command to bring the device out of idle mode. Such is well known in the art. The device is then in active mode. Active mode is communicated to the user, preferably, through an auditory cue. Most preferably, the device will deliver a statement such as “good morning” or “good evening”, said greetings being appropriate given the time of day. In this embodiment, the device includes an internal clock that is electrically connected to the auditory unit allowing the device to give the appropriate greeting. Alternatively, the device can deliver an auditory cue that is time and date independent, such as a chirp or a beep. Further, the cue can be non-auditory, for example, the display unit will light up or an LED indicator will light. A variety of other cues can be used with the current invention and such other cues do not exceed the scope of the current disclosure.

Once the user has activated the device and is properly positioned on the device for measuring, the device will determine whether a reference measurement has been set. Typically, reference measurements are set when the user is using the device of the first time, or if the user has reset the device's memory to erase any previous reference measurements and/or subsequent measurements and trend information. If the device determines that a reference measurement is needed the device will retrieve the measurements and will store these measurements in the device memory labeling the measurements as reference measurements. Once the measurements are obtained, labeled and stored by the device the device will notice the user that measuring is complete. In a preferred embodiment, that notice is auditory and the notice is most preferably a statement such as “thank you, your reference measurement is stored and your next measurement will indicate change in the measurement.” Alternatively, the auditory notice can be a beep or chirp. Further, the auditory notice can be visual such as a printed statement shown on the display screen or a lighted LED. A variety of other notices can be used with the current invention and such other notices do not exceed the scope of the current disclosure.

After these steps, the user can then step off of the device and the device will return to either idle mode or can be powered off, depending on the preference of the user.

If the user positions themselves on the device for measuring and the device determines that there is at least a reference measurement and maybe also measurements subsequent that reference measurement, the device will access the stored information. The device will also obtain the current measurement and will plot the new measurement with respect to the stored information. The device will then indicate to the user difference data. For example, this difference data can be: current measurement minus reference measurement; current measurement minus most recent previous measurement; or current measurement minus selected previous measurement. As a further example the difference data can be indicated to the user as a trend. For example, the trend data can be a graph and the graph can display as a line or bar wherein the x-axis is date and the y-axis is difference. The line can further be point-to-point or best fit. A variety of other means for displaying the difference data can be employed by those of skill in the art and these different means are well within the spirit of the current invention.

Optionally, and as a further embodiment, the device can deliver along with the measurement difference a motivational notice. The motivational notice can be auditory or visual, and comprise such motivational statements like “way to go” or “good job” or can be a cheering or applause sound. The motivational notice can be delivered based on a desired change goal, which is programmed into the device by the user or the cheer can be delivered any time the change in measured data moves in a favorable/desired direction. For the embodiment wherein the desired change goal is programmed into the device by the user, a programming means in provided on said device. Programming means are well known in the art and can include keypads and slots to accept disks and other external media.

Once the difference data has been communicated to the user the user can either step off of the device, allowing the device to store the recently measured data in memory, or the user can reset the device, thus wiping all saved data from memory. Resetting the device will erase the reference measurement and any measurements received by the device subsequent to the reference measurement. Additionally, all comparison data and change/trend information will be erased. Thus, the next time the user decides to use the device the reference measurement will have to be obtained by the device.

To indicate to the device that the user desires to reset, there is a reset means. In the preferred embodiment, the reset means is time based. For example, in one embodiment the device will display to the user the difference data for a defined period of time such as 10 seconds. If the user is still on the device the device will begin delivering an auditory cue, such as a beeping sound. The beeping sound will indicate to the user that the device will automatically reset unless the user steps off of the device. The user is give a period of time to react, such as 5 seconds. If the user remains on the device the device will reset; however if the user steps off of the device the device will not reset.

Alternatively, the device can have a reset switch that is activated by the user to reset the device. The switch is preferably a recessed button located on a surface of the device housing wherein the recessed button is not accidentally depressed during the normal course of measurement obtainment use. These and a variety of other reset means can be employed with the current invention by one of skill in the art. These variations are well within the current disclosure.

With this single user embodiment, there is a risk that a person other than the user (non-user) will step on the device and the device will obtain a measurement from the non-user. The device could then add that contaminating measurement to the user's profile, thereby causing contamination of the data set. Contamination of the data set has a negative impact on the user's data set because the data set comprises data points collected at various times and used to communicate progress or trends to a single user. Non-user data points will factor into the user's data set thereby providing inaccurate representation of the users changing data points over time. One option to prevent use of the device by persons other than the user is that the user put the device away when not in use. This will make the device unlike the typical measurement devices, such as a bathroom scale, that typically sit on the bathroom floor for all to use.

A further option is that the device is equipped with a means for recognizing the authorized user. Such means include, passwords added by a keypad, keys and biometric identification, as well as other means. In the preferred embodiment, the device will comprise a keypad as a recognition means allowing the user to enter a simple password. In this embodiment the keypad can is preferably linked to the device using radio frequency, thereby allowing the user to store the keypad on a shelf where it is easily within reach, while having the device stored on the floor. Alternatively, the keypad can be hardwired into the device and further comprise a support pole or a long flexible wire allowing the keypad to also be stored on a shelf while the device remains on the floor.

In an alternative embodiment, the device comprises a biometric indicator as a recognition means. More preferably, this biometric indicator is a toe-print recognition device, thus allowing the user to step on the device placing their toe in the toe-print recognition device. The device will then scan for identifying features of the user and upon identification will access and update the stored measurement information.

In the embodiments wherein the device includes a recognition means, the user password or other identification must be programmed upon first use. Thus, when the device is obtaining the reference measurement for the first time, and optionally during any subsequent reference measurements, the user must program in a password or other identification. Thus, when the user brings the device from idle mode to active mode, the device will obtain a password or identification and will store this information in connection with the referenced data and the subsequent associated data.

### Example 2

### Multi-User Device

Multi-user devices function similarly to the single-user device described above. The major difference is that the device must compartmentalize saved user data for a variety of users and only obtain, update and present the data for a current user of the device. This is achieved best by using user identification means associated with the measured data, and so the device is preferably equipped with a means for recognizing each user. Such means include, passwords added by a keypad, keys and biometric identification, as well as other means. In this current example, and as is illustrated in FIG. 3b, the device comprises a biometric indicator for identifying the current user. In use, the user first steps on the scale and places his or her toe into the toe print recognition site of the biometric identification means.

As is described above in Example 1, on first use the device determines whether a current user has information that is stored in the device memory. In this example, the device makes this determination based on the biometric information received from the user. If a user is not recognized, the device will request the user set a password or other identification, and then will obtain the user's reference measurements. The device will create a user profile that is independent from any other user profiles that may exist in the memory, and will obtain and store reference measurements within that profile.

Existing users will identify themselves to the device using the biometric identification means. The identified user's measurements will be obtained and stored in that user's profile as data points forming the stored data set. The obtained data point will be compared to other data points in the user's profile and the difference data will be communicated to the user. It is notable that the order of steps can vary: e.g., the device may require the user identification before the measurements are obtained; or the measurements may be obtained before or simultaneously with the user identification information and the measurements held in temporary memory until the user profile is established or accessed and the measured data stored therein.

We claim:

1. A device for obtaining a series of measurements and communicating only the differences between data points in the series of measurements, comprising:
a. a housing;
b. an electronics component further comprising a measuring means and a data processing means; and
c. a communication means.

2. The device of claim 1 wherein said housing is configured as a device for obtaining measurements comprising weight measurement, body mass measurement, fat percentage measurement, bioimpedience measurement, and calorie count measurement.

3. The device of claim 2 wherein the housing is configured to obtain weight measurements, the housing further comprising a weight measuring surface.

4. The device of claim 3 wherein the weight measuring surface is a platform allowing a user to stand thereon and be measured by the device.

5. The device of claim 1 wherein the measuring means comprises means for obtaining weight measurement, body mass measurement, fat percentage measurement, bioimpedience measurement, and calorie count measurement.

6. The device of claim 1 wherein the measuring means obtains measured data and transfers the measured data to the data processing means.

7. The device of claim 6 wherein the data processing means is a microprocessor with memory and wherein the measured data is saved in the memory.

8. The device of claim 7 wherein measured data is processed by the data processing means to compare measured data obtained at different times and wherein the comparison is a difference between these measured data.

9. The device of claim 6 wherein the data processing means is a microprocessor with memory and wherein user specific data is process and stored.

10. The device of claim 9 wherein the user specific data comprises, measurement data, compared measurement data, compared measurement data goals, user identification profile, and user passwords.

11. The device of claim 1 wherein the communication means comprises visual display, liquid-crystal display, light emitting diode, auditory alerts and speakers.

12. The device of claim 11 wherein the communication means is a liquid-crystal display and communicates compared measurement data as a graph showing difference as a function of time.

13. A device for obtaining a series of measurements and communicating only the differences between data points in the series of measurements, comprising:
a. a housing;
b. an electronics component further comprising a measuring means, a data processing means, a programming means, and a recognition means; and
c. a communication means.

14. The device of claim 13 wherein said housing is configured as a device for obtaining measurements comprising weight measurement, body mass measurement, fat percentage measurement, bioimpedience measurement, and calorie count measurement.

15. The device of claim 14 wherein the housing is configured to obtain weight measurements, the housing further comprising a weight measuring surface.

16. The device of claim 13 wherein the measuring means comprises means for obtaining weight measurement, body mass measurement, fat percentage measurement, bioimpedience measurement, and calorie count measurement.

17. The device of claim 13 wherein the measuring means obtains measured data and transfers the measured data to the data processing means.

18. The device of claim 17 wherein the data processing means is a microprocessor with memory and wherein the measured data is saved in the memory.

19. The device of claim 18 wherein measured data is processed by the data processing means to compare measured data obtained at different times and wherein the comparison is a difference between these measured data.

20. The device of claim 13 wherein the recognizing means comprises a password and a keyboard, a key, and a biometric indicator.

21. The device of claim 20 wherein the recognizing means obtains biometric user information and assigns said biometric user information to a user profile.

22. The device of claim 20 wherein the recognizing means is a password and keyboard and the password is assigned to a user profile.

23. The device of claim 13 wherein the programming means comprises, a port to accept floppy disk, CD media, DVD media, or jump drives; a keyboard, and a port to accept broadband, fiber optic, telephone line or wireless communication connections.

24. The device of claim 23 wherein the programming means is useful for transferring and receiving data relating to the obtaining, storage, processing and communicating of measured data points.

25. The device of claim 13 wherein the communication means comprises visual display, liquid-crystal display, light emitting diode, auditory alerts and speakers.

26. A method of obtaining measurements and communicating differences between two or more obtained measurements comprising the steps of:
a. receiving measurement data;
b. determining whether the received measurement data is a first or a subsequent measurement data for the user;
c. processing the received measurement data; and
d. communicating information relating to the measured data to the user.

27. The method of claim 26 wherein the step of receiving measurement data comprises receiving weight measurement data, body mass measurement data, fat percentage measurement data, bioimpedience measurement data, and calorie count measurement data.

28. The method of claim 27 wherein the received measurement data is a first data point in a data set.

29. The method of claim 27 wherein the received measurement data is a subsequent data point in a data set

30. The method of claim 29 wherein the step of processing the data comprises comparing the subsequent data point to the first data point.

31. The method of claim 30 wherein the step of processing the data comprises comparing the subsequent data to an earlier data point that is not the first data point.

32. The method of claim 26 wherein the step of processing the received measurement data comprises generating a value for the difference between different data points in a data set.

33. The method of claim 32 wherein the difference between different data points is communicated to the user.

34. The method of claim 26 wherein the step of communicating information relating to measured data comprises, communicating that the measurement is a first data point, communicating that the measurement is a subsequent data point, communicating the difference between data points, communicating when a measurement goal is reached, communicating progress towards a measurement goal, communicating a user identification, communicating a user identification logon, and communicating instructions, with the proviso that the absolute value of any data point is never communicated to the user.

35. The method of claim 26 wherein the step of communication to the user comprises user profile information to an authorized user.

36. The method of claim 35 wherein the user profile is on a single user device.

37. The method of claim 35 wherein the user profile is on a multiple user device.
